(19) United States
(12) Patent Application Publication
(10) Pub. No.: US 2022/0354698 A1
(43) Pub. Date: Nov. 10, 2022

(54) PLASTIC ALLOY EARPLUG CORE

(71) Applicant: 3M INNOVATIVE PROPERTIES COMPANY, St. Paul, MN (US)

(72) Inventors: Jacob H. Ely, Carmel, IN (US); Robert C. Coffin, Plainfield, IN (US)

(21) Appl. No.: 17/754,022

(22) PCT Filed: Sep. 15, 2020
(86) PCT No.: PCT/IB2020/058568

§ 371 Date: Mar. 22, 2022

Related U.S. Application Data
(60) Provisional application No. 62/904,731, filed on Sep. 24, 2019.

(51) Int. Cl. A61F 11/08; B29B 11/10; B29C 44/06

(52) U.S. Cl. CPC A61F 11/085; B29B 11/10; B29C 44/06; A61F 2240/001; B29K 2995/0002; B29K 2077/00

(57) ABSTRACT

A push-in earplug is provided. The push-in earplug comprises an elongate core comprising a first material and a second material. The push-in earplug also comprises an outer layer comprising a foamable material, the outer layer covering at least a portion of an outer surface of the elongate core. The second material is configured to thermally bond to the outer layer during activation of the foamable material.

### BACKGROUND

The use of hearing protective and noise attenuating devices are well known, and various types of devices have been considered. Such devices include earplugs and semi-aural devices partially or completely constructed of foam or rubber materials that are inserted into, or placed over, the ear canal of a user to physically obstruct the passage of sound waves into the inner ear.

Compressible or “roll-down” type earplugs generally comprise a compressible, resilient body portion and may be made of suitable slow recovery foam materials. The earplug may be inserted into the ear canal of a user by first rolling it between fingers to compress the body portion, then pushing the body portion into the ear canal, and subsequently allowing the body portion to expand to fill the ear canal.

Push-in type earplugs have also been considered, and may include a compressible attenuating portion and a stiff portion that extends from the attenuating portion. To insert a push-in type earplug, the user grasps the stiff portion and pushes the attenuating portion into the ear canal with an appropriate level of force. The attenuating portion compresses as it is accommodated in the ear canal. Push-in earplugs may allow the earplug to be quickly and easily inserted in an ear canal, and may promote hygiene by minimizing contact with the attenuating portion of the earplug prior to insertion.

Although push-in earplugs exhibit desirable characteristics in various applications, they may be costly and may pose difficult manufacturing challenges.

### SUMMARY

A push-in earplug is provided. The push-in earplug comprises an elongate core comprising a first material and a second material. The push-in earplug also comprises an outer layer comprising a foamable material, the outer layer covering at least a portion of an outer surface of the elongate core. The second material is configured to thermally bond to the outer layer during activation of the foamable material.

### BRIEF DESCRIPTION OF THE DRAWINGS

FIG. 1 is a cross-sectional view of a push-in earplug according to the present invention.

FIG. 2 is a microscopic view of a push-in earplug core illustrating a blended polymeric core of an earplug in an embodiment of the present invention.

FIGS. 3A-3D are cross-sectional views of exemplary push-in earplugs according to the present invention showing sound attenuating portions having various exemplary shapes.

FIG. 4 is a perspective view of a pre-form that includes an elongate core and an outer layer in an intermediate state of an exemplary method of making an earplug.

FIG. 5 is a schematic representation of an exemplary manufacturing process according to the present invention.

FIGS. 6A and 6B are cross-sectional views of an example of a mold used in an exemplary embodiment of the present invention.

FIGS. 7A-7B are cross-sectional views of an example of a mold used in an exemplary embodiment of the present invention.

FIG. 8 is a schematic representation of an exemplary manufacturing process according to the present invention.

FIG. 9 is a method of manufacturing a push-in earplug in an exemplary embodiment of the present invention.

FIGS. 10A and 10B illustrate earplugs and preforms as discussed in the Examples.

### DETAILED DESCRIPTION

“Mold” means a hollow form that may or may not impart a shape on a component placed in the hollow form.

“Thermally bonded” means a state in which molecules of two materials or surfaces have diffused into the material or surface of the other when in a molten phase such that a bond is formed. Chemical bonding is absent or does not provide the primary source of bonding between thermally bonded materials or surfaces.

“Thermoplastic” means a polymer that can be repeatably heated and re-shaped and will retain its shape upon cooling.

“Thermoset” means a polymer that may be irreversibly cured.

“Unactivated” when referring to a foaming agent means that the foaming agent can be further activated to facilitate the formation of gas or cells in a material.

Unless otherwise indicated, all compositions are presented as weight percentages.

An earplug that provides hearing protection for a user, and a method of making an earplug, is provided in the following description. Earplugs as described herein includes a relatively stiff elongate core covered, directly or indirectly, by a relatively soft outer layer. The outer layer includes a compressible sound attenuating portion that may be inserted into the ear canal of a user, and stem portion that may be grasped by a user to handle the earplug. Such an earplug may be easily inserted into an ear canal without first requiring that the sound attenuating portion be compressed or “rolled down.”

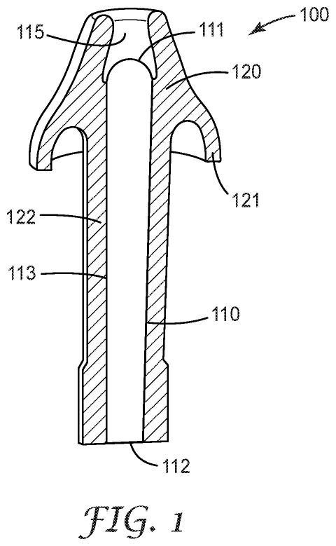

FIG. 1 is a cross-sectional view of a push-in earplug according to the present invention. Earplug 100 is a push-in earplug with an elongate core 110 having first and second ends 111 and 112, and an outer major surface 113. Earplug 100 further includes an outer layer 120 bonded, directly or indirectly, to at least a portion of outer major surface 113 of elongate core 110. Outer layer 120 includes a sound attenuating portion 121 for at least partial insertion into the ear canal of a user, for example, and a stem portion 122 having a smaller diameter and greater average density than sound attenuating portion 121.

During insertion of earplug 100, stem portion 122 and elongate core 110 serve as a handle which may be gripped by a user. Earplug 100, and specifically sound attenuating portion 121, is brought proximate to the user's ear and inserted into the ear canal. Sound attenuating portion 121 compresses as it is positioned, and elongate core 110 provides sufficient stiffness to facilitate insertion. In use, sound attenuating portion 121 is positioned substantially within an ear canal to block the passage of sound and stem portion 122 extends outwardly from the ear canal to provide a handle to remove the earplug.

In one embodiment, elongate core 110 has a circular cross-section that is substantially uniform at any location between first and second ends 111 and 112 such that elongate core 110 exhibits a generally cylindrical shape. A circular cross section may minimize edges that may cause discomfort by contacting portions of a user's ear. In various other exemplary embodiments, elongate core may have a triangular, square, or other suitable cross-section, or may have a cross-section that varies along the length of earplug 100. Outer major surface 113 may have a knurled, grooved, or otherwise textured surface. Such a surface may increase the surface area that contacts outer layer 120 or an intermediate layer such that a robust bond is created. In some embodiments, elongate core 110 includes multiple concentric layers, such as a layer to provide a desired stiffness and a layer that facilitates a robust bond with the outer layer, or that provides other desired characteristics.

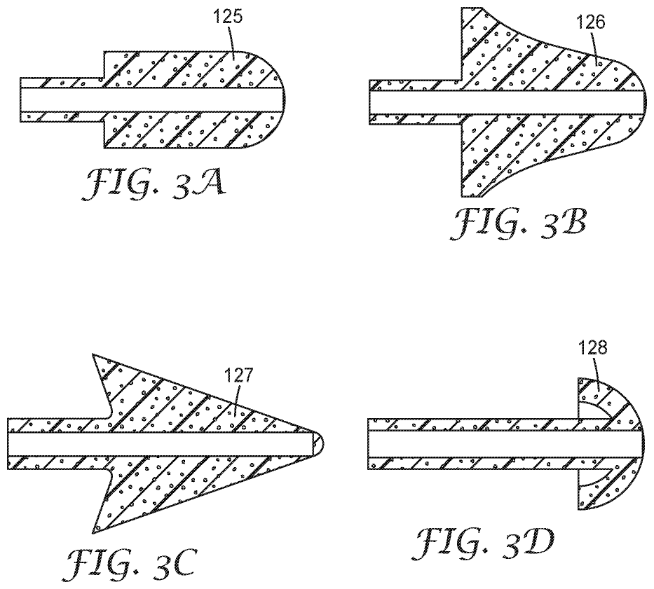

Earplug 100 further includes an outer layer 120 substantially covering, directly or indirectly, elongate core 110. Outer layer 120, in one embodiment, includes both a sound attenuating portion 121 and stem portion 122. In one embodiment, outer layer 120 substantially surrounds outer major surface 113 of elongate core 110 and extends from first end 111 to second end 112 of elongate core 110. In some embodiments, outer layer 120 is a contiguous layer such that portions of sound attenuating portion 121 contact portions of stem portion 122. First and second ends 111 and 112 of elongate core 110 may be at least partially exposed, and elongate core 110 may be colored similarly or dissimilarly from the color of outer layer 120 to hide or exhibit the presence of elongate core 110. Sound attenuating portion 121 is positioned near first end 111 of elongate core 110 and is shaped to be accommodated in an ear canal of a user. In one embodiment, sound attenuating portion 121 has a bell-shape, and has a diameter at its widest point that is greater than a diameter of stem portion 122. In various other embodiments shown in FIGS. 3A through 3D, for example, sound attenuating portions 125, 126, 127, 128, respectively, may be bullet-shaped, hemispherical-shaped, cone-shaped, mushroom-shaped, or otherwise shaped to provide a desired fit or to suit a particular application.

Outer layer 120, as described in greater detail below, is formed of a material that is configured to, when heated, expand to fill a mold, allowing for the creation of a variety of shapes for sound attenuating portion 121. The material of outer layer 120 may be selected to control the friability of the outer layer 120 such that it may not easily be broken or disintegrate during use. The friability of an earplug may be controlled in part by selecting a material having an appropriate molecular weight, with higher molecular weight generally resulting in a less friable earplug.

The density of outer layer 120 can be controlled during manufacturing to provide a specified density as desired for a particular application. Outer layer 120 may exhibit a density that varies by thickness, for example, such that outer layer 120 has an integral outer skin that is more dense than the remainder of outer layer 120. Such a skin may be present on one or both of sound attenuating portion 121 and stem portion 122. Alternatively, sound attenuating portion 121 or stem portion 122 may have a substantially uniform density. In some embodiments, outer layer 110 is a foamable thermoplastic elastomer. The thermoplastic elastomer may be styrene-ethylene-butadiene-styrene (SEBS), a styrene-isoprene rubber (SIS), or a combination thereof.

Elongate core 110 provides a substrate which outer layer 120 may cover, directly or indirectly, and facilitates insertion of earplug 100 into the ear canal of a user. Elongate core 110 needs to have greater rigidity or stiffness than outer layer 120 but should be soft enough to be comfortable and safe for a user. Elongate core 110 should provide sufficient rigidity that earplug 100 may be positioned for use at least partially in the ear of a user by pushing sound attenuating portion 121 into the ear canal with an appropriate force. A sufficiently stiff elongate core 110, combined with an appropriate outer layer 120, will allow earplug 100 to be positioned at least partially in the ear of a user without the need to first compress or “roll down” sound attenuating portion 121. Direct insertion without the need to first compress or “roll down” sound attenuating portion 121, for example, promotes hygiene by limiting contact with sound attenuating portion 121 prior to placement in the ear. Elongate core 110 should also exhibit an appropriate level of flexibility such that it may slightly deform to the contours of the ear canal when positioned for use.

One important aspect of earplug design and construction, therefore is the selection of materials for elongate core 110 and outer layer 120. During the manufacturing process, the foamable outer layer 120 will heat up and expand to fit the shape of a mold. At the same time, the foamable layer 120 will also thermally bond to core 110. This requires at least some miscibility with the thermoplastic elastomer.

The core material should not melt or deform at the temperatures required for expansion of foamable overcoating layer 120. However, it is desired that elongate core 110 be made of a material that will thermally bond with outer layer 120. Elongate core 110 needs to experience some blending with the outer layer 120 during manufacturing, but also maintain its own structural integrity. Additionally, it is desired that the core material have a tunable stiffness, such that the earplug is stiff enough for insertion into a user's ear, but still comfortable during use.

Several different materials were explored as possible materials for a suitable elongate core 110. Polyolefins are miscible with many suitable thermoplastic elastomers. Miscibility is important as the separate core 110 and outer layer 120 will not experience chain entanglement, and surface forces (van der Waals forces) will not be sufficient to bond the layers together. Polypropylene was selected for its higher heat tolerance. A polypropylene core experienced too much deformation and did not provide sufficient support during the molding process. Filling the polypropylene core with mineral-based filler material provided additional support, but resulted in a core that was too stiff. Other polymer bases were also explored, including acrylic, acrylonitrile butadiene styrene (ABS), polyurethane and others.

A third layer was also explored to add structure, however this would add an additional step to the manufacturing process, increasing costs and complexity.

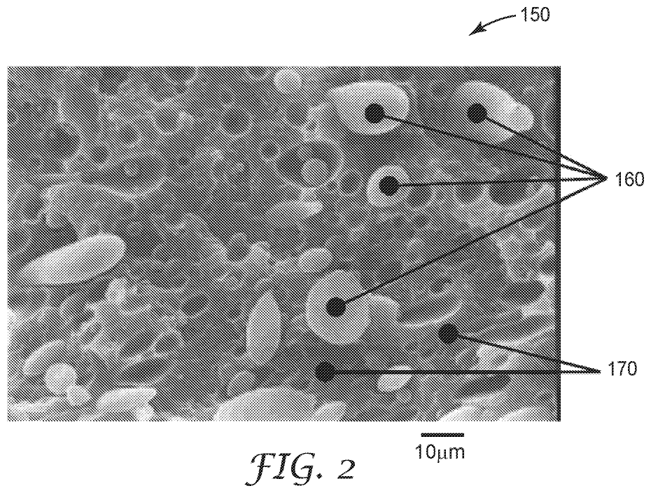

FIG. 2 is a microscopic view of a push-in earplug core illustrating a blended polymeric core of an earplug in an embodiment of the present invention. Blended core 150 has a polyamide matrix 170 which supports a plurality of polypropylene domains 160.

A blend of polypropylene and polyamide was found to provide sufficient structure during the manufacturing process while also allowing thermal bonding between the core and the foamable overcoating layer. Polyamide forms lattice structure 170 with a high melting supporting scaffold, with a melting temperature around 220° C. Polypropylene domains 160 facilitate bonding between outer layer 120 and core 110. Polyamide has a high enough melting temperature to remain solid during the foaming step, allowing thermal bonding between polypropylene domains 220 and outer layer 120.

In some embodiments, core 110 comprises at least about 50% of a polyamide mixture, or at least about 55% or at least about 60%. In some embodiments, core 110 comprises about a 65% polyamide mixture and about 35% polypropylene. The polyamide mixture comprises an impact modifier. The impact modifier is at least 10% or at least 15% or at least 20% or at least 25% of the polyamide mixture. In one embodiment, the impact modifier is maleated styrene-ethylene-butylene-styrene (SEBS). Inclusion of an impact modifier resulted in an improved flexibility, but surprisingly did not result in a significant reduction in core stability. It was expected that the inclusion of an impact modifier would result in a lower heat deflection temperature, resulting in greater deformation when heated in a mold. However the Embodiments described herein have similar bond quality as a pure polypropylene core without the deformation in manufacturing. Cord-core pull strengths also improved from 2 lbs to 6 lbs with the addition of an impact modifier. Cord bonding strength must be above a minimum value to prevent unwanted/accidental cord detachment. Cord-core pull strength is measured in a tensile test machine such as a Model 5967 Universal Testing System, available from Instron, Inc. (Norwood, Mass., USA). The cord is secured in one clamp while the plug is secured in an opposing clamp. The clamps move apart at a given rate, while the pull force is measured with a load cell. The test stops when the force drops off sharply. The peak force is recorded.

FIGS. 3A-3D are cross-sectional views of exemplary push-in earplugs according to the present invention showing sound attenuating portions having various exemplary shapes. While FIG. 1 illustrates one example shape of a sound attenuating portion of a push-in earplugs, other embodiments are shaped differently. For example, any of sound attenuating portion shapes 125, 126, 127 or 128 may be possible. Other suitable shapes are also envisioned.

FIG. 4 is a perspective view of a pre-form that includes an elongate core and an outer layer in an intermediate state of one method of making an earplug. Earplug 100 may be formed in a multiple step process. In one embodiment, earplug 100 is formed in a process that involves an intermediate state in which outer layer 120 is covered around elongate core 110, directly or indirectly, to result in a pre-formed hearing protection device such as pre-form 130, but does not yet include sound attenuating portion 121.

In the intermediate state shown in FIG. 4, outer layer 120 of pre-form 130 includes an unactivated foaming agent. In one embodiment, the unactivated foaming agent includes an expandable sphere foaming agent that includes thermoplastic spheres, for example, that include a shell encapsulating a hydrocarbon or other appropriate gas that expands when exposed to heat or other activation source. Expansion of the thermoplastic shell results in an increased volume and reduced density of the material of outer layer 120. The unactivated foaming agent may also be a chemical foaming agent that includes an expandable material that is self-contained or otherwise not contained by an expandable sphere. Activation of such a foaming agent causes the expandable material to expand, creating voids or gaps in the material of the outer layer. In one embodiment, outer layer 120 of pre-form 130 includes an unactivated expandable sphere foaming agent and an unactivated chemical foaming agent. Activation of the foaming agent or agents present in outer layer 120, and the associated expansion of outer layer 120, can be controlled to provide an earplug 100 having a sound attenuating portion 121 and stem portion 122 exhibiting a desired shape, density, hardness, and other desired characteristics.

The presence of both an expandable sphere foaming agent and a chemical foaming agent may assist in providing sufficient structure and expansion such that outer layer 120 may be appropriately formed during activation, while reducing the hardness of outer layer 120 from a level that would otherwise result if only an expandable sphere foaming agent were used. Some or all of a gas generated by a chemical foaming agent may escape during activation such that some or all of the gas is not present in the outer layer after activation. Some or all of an expandable sphere foaming agent may remain in the outer layer of a final earplug such that a final earplug may include thermoplastic spheres. In one embodiment, outer layer 120 of earplug 100 includes between 1% and 5% weight, and may include approximately 3% weight, of the foaming agent or remnants of the foaming agent.

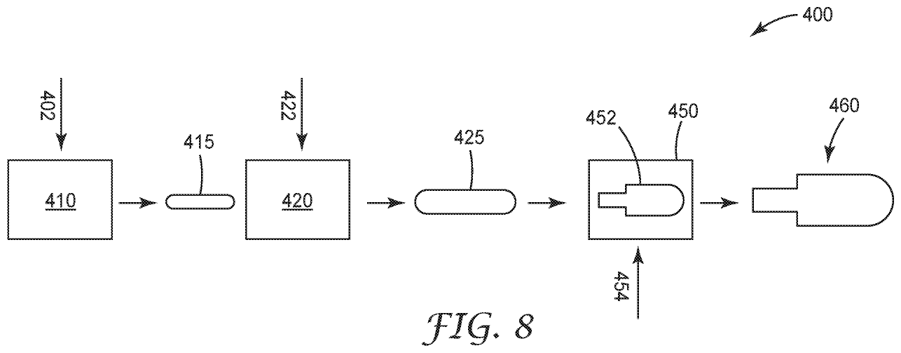

In the intermediate state shown in FIG. 4, pre-form 130 may be cut to the desired length of earplug 100, may be cut to an extended length sufficient for subsequent formation of many earplugs, or may remain uncut such that activation of outer layer 120 occurs prior to cutting as described below with reference to FIG. 8. Pre-form 130 having an extended length may facilitate handling for subsequent processing and activation of the foaming agent. In one embodiment, pre-form 130 is cut to an extended length that can be subsequently cut and activated to yield a desired quantity of earplugs 100. An extended pre-form 130 may be coiled or otherwise shaped for ease in transporting or handling.

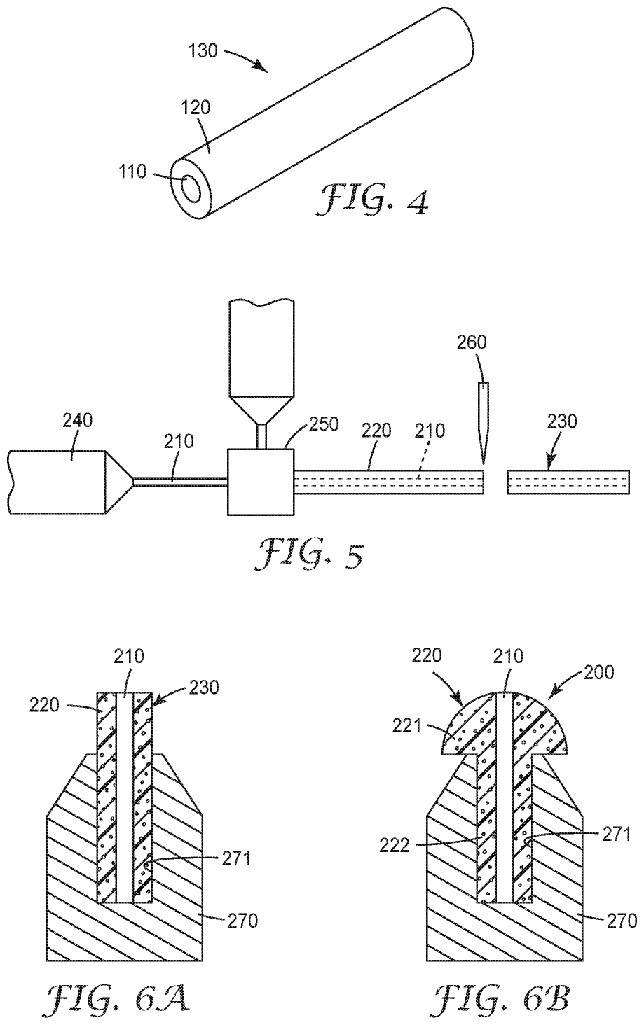

FIG. 5 is a schematic representation of one manufacturing process according to the present invention. The present invention further provides a method of making an earplug. The method may include the steps of covering a substrate, such as a blended polymeric elongate core, with an outer layer that includes an unactivated foaming agent, and activating the foaming agent such that at least a portion of the outer layer expands into a desired shape.

The present invention provides a method of making personal protective equipment, such as earplug 100 described with respect to FIG. 1. One method includes steps of covering a substrate with an outer layer, and applying heat to at least a portion of the outer layer such that at least a portion of the outer layer expands. Expansion of the outer layer occurs due to activation of a foaming agent present in the material of the outer layer and can be controlled by positioning at least a portion of the outer layer in a mold prior to expansion. Portions of the outer layer may be confined by the shape of the mold as the outer layer expands, or are shielded from heat to limit activation of the foaming agent.

The method described herein is suitable not only for manufacturing earplugs, but also for manufacturing other types of hearing protection devices and components for other personal protective equipment, as well as other molded or formed parts suitable for other applications. For example, the present method provides a process for making a seal for a facepiece of a respiratory protection device that can be foamed to provide a desired shape and density. Other exemplary applications include the manufacture of ear muffs, respirators, eyewear, other personal protective equipment, components of such personal protective equipment, and other applications.

One method of making a push-in earplug according to the present invention includes the steps of covering a blended elongate core, directly or indirectly, with an outer layer comprising an unactivated foaming agent, and activating the foaming agent of at least a portion of the outer layer to form a sound attenuating portion and a stem portion bonded, directly or indirectly, to the blended elongate core.

The blended elongate core, in one embodiment, comprises a polyolefin. In one embodiment, the blended elongate core comprises an impact modifier. In one embodiment, the blended elongate core comprises at least about 50% polyamide, or at least about 60% polyamide, or at least about 65% polyamide. The blended core comprises, in one embodiment, at least 10% of a bonding component, or at least about 15% of a bonding component, or at least about 20% of a bonding component, or at least about 25% of a bonding component, or at least about 25% of a bonding component, or at least about 30% of a bonding component, or at least about 35% of a bonding component. In one embodiment, the bonding component is polypropylene.

The polyamide mixture, in one embodiment, comprises at least 10% impact modifier, or at least 15% impact modifier, or at least 20% impact modifier, or at least 25% impact modifier. In one embodiment, the impact modifier is SEBS.

FIG. 5 shows a schematic of one method of making an earplug 200 according to the present invention. An extended blended elongate core 210 is formed by extruding a first material through a first die 240 and drawing the first material to an appropriate diameter. The blended elongate core may be solid or may include a longitudinal channel extending through all or a portion of elongate core 210, and may include one or more concentric layers having differing characteristics. After extrusion, the blended elongate core may be cooled such that it remains stable in subsequent steps of the manufacturing process. The magnitude of temperature change may depend on the materials used and the desired characteristics of the final product. In one embodiment, elongate core 210 is cooled as necessary such that it exhibits a temperature at a point before being covered by second die 250 that is lower than an activation or curing temperature of outer layer 220. Prior to being covered, blended elongate core 210 has an extended length and is not yet cut to the desired length for an earplug.

In the embodiment shown in FIG. 5, blended elongate core 210 is covered, directly or indirectly, with an outer layer 220 comprising a foamable material, by second die 250. Second die 250 may be a co-extrusion die or other suitable die as known in the art. In one embodiment, the foamable comprises a thermoplastic and one or more unactivated foaming agents. Outer layer 220 is applied to blended elongate core 210 while remaining at a temperature below an activation temperature of the unactivated foaming agents. In one embodiment, outer layer includes SEBS and a foaming agent having an activation temperature between 100° C. and 205° C., 120° C. and 190° C., or of about 170° C. Other suitable materials include plasticized polyvinyl chloride, ethylene propylene diene monomer (EPDM) rubber, styrene butadiene rubber (SBR), butyl rubber, natural rubbers, other thermoplastics, thermoset polymers, and other suitable materials as known in the art. In embodiments in which outer layer 220 includes a second material having a rubber or thermoset polymer, outer layer 220 may be applied at a temperature below a vulcanizing or curing temperature of the rubber or thermoset polymer. In such an embodiment, outer layer 220 may include an unactivated foaming agent and an uncured or partially cured rubber or thermoset polymer that can be subsequently activated and cured, respectively, with heat or other suitable activation or curing process.

The weight percentage of foaming agent in outer layer 220 when initially applied to blended elongate core 210 may be selected based on the type of thermoplastic or other material used and the desired final shape, density, hardness or other characteristics of sound attenuating portion 221. In one embodiment, outer layer 220 has an initial composition of between 90% and 99.5% SEBS and between 10% and 0.5% of an appropriate unactivated foaming agent, or of approximately 93% SEBS and 7% of an unactivated expandable sphere foaming agent, such as EXPANCEL 930 DU 120, EXPANCEL 920 DU 120, both available from Eka Chemicals AB of Sundsvall, Sweden.

In other embodiments, outer layer 220 has an initial composition including an unactivated chemical foaming agent such as oxybis benzene sulfonyl hydrazide (OBSH) available from Biddle Sawyer Corp. of New York, N.Y. The presence of a chemical foaming agent such as an OBSH foaming agent may yield a sound attenuating portion having a lower hardness value than a sound attenuating portion formed of an outer layer including an expandable sphere foaming agent such as EXPANCEL as the only foaming agent.

In one embodiment, outer layer 220 includes an unactivated expandable sphere foaming agent and an unactivated chemical foaming agent. The presence of both an expandable sphere foaming agent and a chemical foaming agent may assist in providing sufficient structure such that the outer layer may be appropriately formed and that may not be present with a chemical foaming agent alone, while reducing the hardness of the outer layer from a level that would otherwise result if only an expandable sphere foaming agent were used. Accordingly, the combination of a chemical foaming agent and an expandable sphere foaming agent may result in an outer layer having a hardness level appropriate for a desired application, such as for insertion into an ear canal. In one embodiment, outer layer 220 when initially applied may include between approximately 0.5% weight and 3% weight of an unactivated chemical foaming agent, or of approximately 2% weight of an unactivated chemical foaming agent, and between approximately 0.5% weight and 9.5% weight of an unactivated expandable sphere foaming agent, or of approximately 2% weight of an unactivated expandable sphere foaming agent. Outer layer 220 may also include other suitable foaming agents, or various combinations of EXPANCEL foaming agents, OBSH foaming agents, and other suitable foaming agents. Outer layer 220 may further include pigment to impart a desired color, antioxidants, UV stabilizers, and oils or waxes to aid in extrusion and mold release as known in the art.

In some exemplary embodiments, outer layer 220 is in a molten state when covered over blended elongate core 210. As a result, molecules of outer layer 220 and elongate core 210, or of one or more intermediate layers, are believed to diffuse into the material or surface of each other and a thermal bond is formed. When the materials or surfaces cool and solidify, outer layer 220 remains thermally bonded, directly or indirectly, to elongate core 210. In one embodiment, significant chemical bonding is absent such that the primary source of bonding between elongate core 210 and outer layer 220 is thermal bonding. In other exemplary embodiments, outer layer 220 contacts elongate core 210 or one or more intermediate layers when covered over elongate core 210 but no significant bond is formed between outer layer 220 and elongate core 210 or one or more intermediate layers. Upon activation and/or curing of outer layer 220, a thermal bond may be formed, directly or indirectly, between outer layer 220 and elongate core 210.

In other exemplary embodiments, elongate core 210 may be covered with outer layer 220, or one or more intermediate layers, by laminating, molding, spraying, dipping, or other suitable process as known in the art as an alternative or in addition to second die 250. Such steps may occur before or after elongate core 210 is cut to a desired length. Regardless of the process used, the temperature of outer layer 220 should remain below the activation temperature of the foaming agent(s) such that the foaming agent(s) remain unactivated during the covering process. In the event that an uncured or partially cured material is included in outer layer 220, such as an EPDM rubber or thermoset polymer, the temperature of outer layer 220 should remain below the curing temperature of the material.

In one embodiment, blended elongate core 210 covered by outer layer 220 is cut to the length of a desired earplug with cutter 260. The result is pre-form 230 having blended elongate core 210 and outer layer 220 in which outer layer 220 includes an unactivated foaming agent that may be subsequently activated to create an earplug having a sound attenuating portion 221 and a stem portion 222.

Cutter 260 may cut pre-form 230 to a desired length of earplug 200, or to an extended length sufficient for subsequent formation of many earplugs. In one embodiment, pre-form 230 is cut to an extended length that can be subsequently cut and activated, or vice versa, to yield a desired quantity of earplugs 200. An extended pre-form 230 may be coiled or otherwise shaped for ease of handling or transportation.

In one embodiment, the unactivated foaming agent present in outer layer 220 includes thermoplastic spheres encapsulating a hydrocarbon or other expandable material. Application of an appropriate amount of heat causes the thermoplastic shell and hydrocarbon to expand. In other exemplary embodiments, the foaming agent includes, alone or in combination with an expandable sphere foaming agent, an expandable material that is self-contained or not otherwise encapsulated, and that produces gas when exposed to heat or other activation source. If left unrestrained, activation of the foaming agent(s) creates cells in outer layer 220, ultimately increasing volume and decreasing density of outer layer 220. Expansion of outer layer 220 can be controlled by the thickness and composition of outer layer 220, selective application of heat, catalyst, or other activation source, and/or by placing at least a portion of pre-form 230 in a mold to limit expansion of outer layer 220 as the foaming agent is activated.

In the exemplary method shown in FIGS. 6A and 6B, mold 270 is used to control expansion of outer layer 220. Mold 270 includes a first cavity 271 in the form of a stem portion that receives a portion of pre-form 230. Pre-form 230 may be cut to the length of a desired earplug 200 prior to being placed in mold 270. Alternatively, pre-form 230 may be of an extended length and may be cut to length after being inserted into mold 270. Cutting pre-form 230 after insertion into mold 270 may facilitate handling and insertion. Heat is applied to the exposed portion of pre-form 230 to raise the temperature of outer layer 220 at least to an activation temperature of a foaming agent present in outer layer 220 and cause outer layer 220 to expand, as shown in FIG. 6B. The portion of earplug 200 positioned in first cavity 271 may be effectively shielded from heat such that activation of the foaming agent is limited. Alternatively, or in addition, first cavity 271 constrains outer layer 220 and substantially inhibits expansion caused by activation of the foaming agent that would otherwise result in a greater volume and less dense outer layer. Blended elongate core 210 and outer layer 220 are subsequently cooled and ejected from mold 270. The finished earplug 200 includes a sound attenuating portion 221 formed by the exposed outer layer that could freely expand and a stem portion 222 that was partially constrained in mold 270 during activation of the foaming agent. Due to the constraint of the mold and/or limited activation of the foaming agent, stem portion 222 may have a greater average density and/or a greater hardness than that of sound attenuating portion 221.

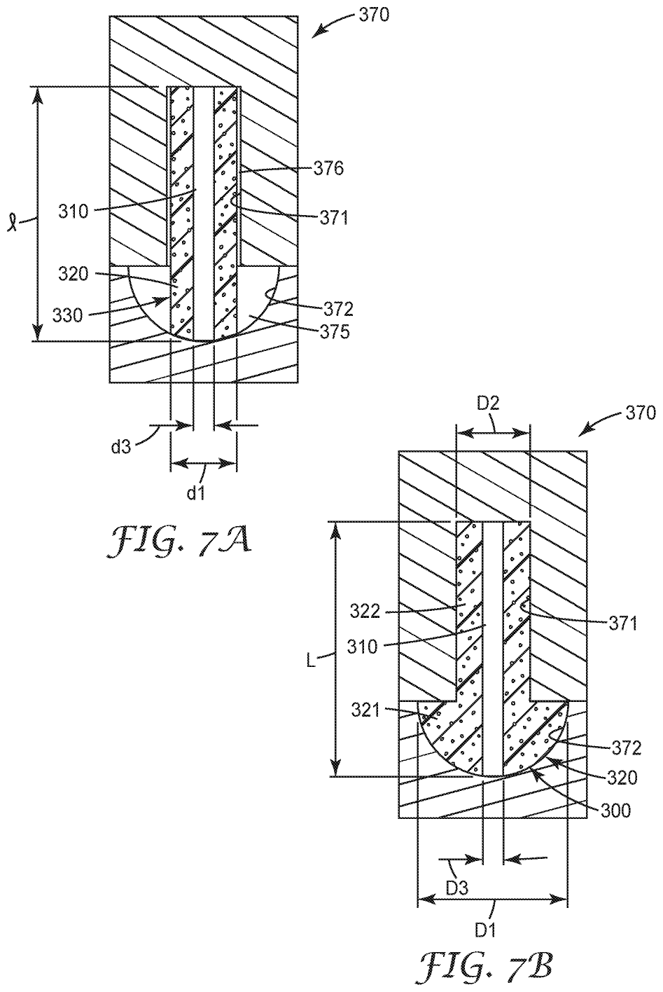

In the exemplary embodiment of FIGS. 7A and 7B, mold 370 is used to control expansion of outer layer 320 of pre-form 330. Mold 370 includes a first cavity 371 in the form of a stem portion that receives a portion of pre-form 330. Mold 370 further includes a second cavity 372 in the form of a sound attenuating portion. When pre-form 330 is initially placed in mold 370, a gap 375 exists between pre-form 330 and a perimeter of second cavity 372. In some embodiments, a small gap 376 may exist between pre-form 330 and a perimeter of first cavity 371. Upon application of heat or other suitable activation source, a portion of outer layer 320 expands to fill gap 375 and substantially conforms to the shape of second cavity 372. The portion of earplug 300 positioned in first cavity 371 may be effectively shielded from heat such that activation of the foaming agent is limited. Alternatively, or in addition, expansion of outer layer 220 that would otherwise occur during activation of the foaming agent is constrained by first cavity 371. Further, as application of heat softens outer layer 320 and the foaming agent is activated, outer layer 320 may expand to fill first cavity 371 and some of outer layer 320 initially in first cavity 371 may flow into second cavity 372 to fill gap 375. In one embodiment, mold 370 includes small gas vents to allow excess gas to escape while preventing passage of any molten material.

In one embodiment, mold 370 is oriented such that first cavity 371 is oriented above second cavity 372 during a portion or all of the activation process. Such an orientation may allow material to flow from first cavity 371 into second cavity 372 during activation. Further, an orientation in which first cavity 371 is oriented above second cavity 372 may facilitate the formation of an integral skin on sound attenuating portion 321 because cells or gaps formed during activation of the foaming agent may tend to move upward and away from a lower surface of cavity 372.

Earplug 300 is subsequently cooled and ejected from mold 370. Finished earplug 300 includes a sound attenuating portion 321 having the shape of second cavity 372 of mold 370, and a stem portion 322 having the shape of first cavity 371 of mold 370. Due to the constraint of first cavity 371 and/or limited activation of the foaming agent in the area of first cavity 371, stem portion 322 may have a greater average density and/or hardness than that of sound attenuating portion 321.

In the exemplary embodiment shown in FIGS. 7A and 7B, earplug 300 is formed from pre-form 330 having a total length l in a longitudinal direction between approximately 15 mm and 40 mm, or of about 25.5 mm. Outer layer 320 has an outer diameter d1 between approximately 2.5 mm and 6.5 mm, or of about 4.5 mm, elongate core 310 has an outer diameter d3 between approximately 1.5 mm and 3.5 mm, or of about 2.5 mm. After activation of outer layer 320 described above, as shown in FIG. 7B, final earplug 300 has a total length L in a longitudinal direction between approximately 15 mm and 40 mm, or of approximately 25.5 mm, sound attenuating portion 321 has an outer diameter D1 at its widest point between approximately 8 mm and 16 mm, or of approximately 12.5 mm, stem portion 322 has a diameter D2 between approximately 3 mm and 10 mm, or of approximately 6.5 mm, elongate core 310 has an outer diameter D3 between approximately 1.5 mm and 3.5 mm, or of approximately 2.5 mm. The dimensions of pre-form 330 and finished earplug 300 can be varied based on the materials of outer layer 320 and elongate core 310, and as required to form a final earplug 300 having desired characteristics for a particular application. While not shown in FIGS. 7A and 7B, it is also expressly contemplated that, in some embodiments, a channel may also be present that extends partly, or completely, from a first end of an earplug to a second end of an earplug.

FIG. 8 is a schematic representation of an exemplary manufacturing process according to the present invention. Components of schematic 400 are not drawn to scale.

A first material 402 is provided to a first extruder 410, which outputs an extruded elongate core 415. Elongate core 415, along with a second material 422, is provided to a second extruder 422, which outputs a preform 425. Preform 425 includes elongate core with an overlayer formed from the second material. Second material 422 may comprise a foamable material.

Preform 425 is provided to a mold cavity 452 within a mold 450. Heat 454, or another activation mechanism, is applied, causing the foamable over layer to expand within, and conform to the shape of, mold cavity 452. Once the activation process is complete, push-in earplug 460 is removed from mold 450.

Extruders 410 and 420 may be configured to draw materials 402, 422 to an appropriate diameter. Either of extruders 410 and 420 may also include a cutting mechanism configured to cut the extruded material to the desired length for an in-ear plug. However, in another embodiment the cutting mechanism (not shown) is a separate operation that takes place before preform 425 is provided to mold 450.

While extruders are illustrated as the mechanism for forming core 415 and preform 425, it is also envisioned that core 415 can be covered with an outer layer using laminating, molding, spraying, dipping or any other suitable process known in the art.

Blended elongate core 415 and the coated core may be cooled in between extrusion and/or cutting operations. Illustrated in FIG. 8 is an embodiment where the entirety of preform 425 is placed within mold cavity 452. However, in some embodiments only a portion of preform 425, the sound-attenuating portion for example, is placed into the mold. In one embodiment, the foaming agent is activated by heat or other activation source to cause the outer layer of preform 425 to expand. In some embodiments in which the outer layer includes an uncured or partially cured material, application of heat or other activation source also causes the outer layer to cure. In one embodiment, mold cavity 452 includes a first cavity in the form of a stem portion and a second cavity in the form of a sound attenuating portion. Upon application of heat or other suitable activation source, a portion of the outer layer expands to fill the second cavity and substantially conform to the shape of second cavity, while the portion positioned in the first cavity is effectively shielded from heat such that activation of the foaming agent is limited. Alternatively, or in addition, expansion of the outer layer that would otherwise occur during activation of the foaming agent is substantially constrained by the shape of the first cavity. In one embodiment, mold 450 includes small gas vents to allow excess gas to escape while preventing passage of any molten material.

In another exemplary embodiment, only a portion of preform 425 is positioned in a mold cavity. The mold cavity may be in the form of a stem such that expansion of a portion of preform 425 is substantially constrained to form a stem, while the remaining portion of preform may freely expand to form a sound attenuating portion. Alternatively, the mold cavity may be in the form of a sound attenuating portion such that expansion of a portion of preform is constrained and selectively activated to form the sound attenuating portion, while the remaining portion of preform 425 is not activated, or is only partially activated, and forms a stem.

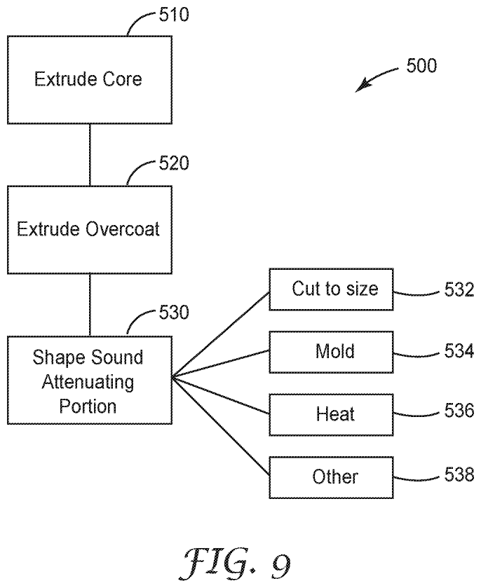

FIG. 9 is a method of manufacturing a push-in earplug in an exemplary embodiment of the present invention. Method 500 may be useful for forming any of the in-ear plugs described herein. Method 500 may be performed using a system similar to the schematic described with respect to FIG. 8, or using another suitable system.

In step 510, a core is extruded. In some embodiments, core 510 comprises at least about 50% of a polyamide mixture, or at least about 55% or at least about 60%. In some embodiments, core 110 comprises about a 65% polyamide mixture and about 35% polypropylene. The polyamide mixture comprises an impact modifier. The impact modifier is at least 10% or at least 15% or at least 20% or at least 25% of the polyamide mixture. In step 520, an overcoat layer is extruded over the core. In one embodiment, the overcoat layer substantially covers the elongate core. The overcoat layer is formed of a material that is configured to, when heated, expand to fill a mold cavity. The material may be selected to control the friability of the outer layer, such that it is not easily broken and does not disintegrate during use. The friability of an earplug may be controlled in part by selecting a material having an appropriate molecular weight, with higher molecular weight generally resulting in a less friable earplug. In some embodiments, the overcoat layer is a foamable thermoplastic elastomer. The thermoplastic elastomer may be styrene-ethylene-butadiene-styrene (SEBS), a styrene-isoprene rubber (SIS), or a combination thereof.

In step 530, a sound attenuating portion is formed. This may comprise cutting the coated core down to size, as indicated in block 532. The sound attenuating portion may be formed by placing at least a portion of the coated core in a mold, as indicated in block 534. The mold may include a cavity with the desired shape of a sound attenuating portion, such as the shapes illustrated in FIGS. 3A-3D, for example, as well as other suitable shapes. The formation of the sound attenuating portion may include activating the overcoat layer with heat, as indicated in block 536. Other processing steps may also be applied, as indicated in block 538. For example, the outer layer may undergo some curing.

An earplug and a method of making an earplug describe herein provides several benefits. The earplug described herein may be comfortably positioned in the ear canal of a user to provide a desired level of hearing protection, and the presence of a stiffer elongate core promotes hygiene by eliminating the need to roll down a sound attenuating portion prior to insertion. The method described herein allows an earplug to be efficiently manufactured. An earplug having an outer layer bonded, directly or indirectly, to an elongate core as described herein eliminates the cost and complexity of an additional step of joining a rigid component to a sound attenuating component required of many prior push-in type earplugs. The elongate core and outer layer can be thermally bonded without the need for an additional adhesive or additional assembly step.

The present invention has now been described with reference to several embodiments thereof. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the exact details and structures described herein, but rather by the structures described by the language of the claims, and the equivalents of those structures. Any feature or characteristic described with respect to any of the above embodiments can be incorporated individually or in combination with any other feature or characteristic and are presented in the above order and combinations for clarity only.

### Embodiments

Embodiment 1 is a push-in earplug. The push-in earplug has an elongate core which has a first material and a second material. The push-in earplug also has an outer layer comprising a foamable material, the outer layer covering at least a portion of an outer surface of the elongate core. The second material is configured to thermally bond to the outer layer during activation of the foamable material.

Embodiment 2 includes the features of embodiment 1, however the foamable material comprises a foaming agent that, when activated, increases volume of outer layer.

Embodiment 3 includes the features of embodiment 2, however the foaming agent comprises an unactivated expandable sphere foaming agent.

Embodiment 4 includes the features of embodiment 2, however the foaming agent comprises unactivated chemical foaming agent.

Embodiment 5 includes the features of any of embodiments 2-4, however the foaming agent is heat activated.

Embodiment 6 includes the features of embodiment 5, however the foaming agent is heat activated at a temperature that allows for thermal bonding with the second material.

Embodiment 7 includes the features of embodiment 6, however the temperature is less than a melting temperature of the first material.

Embodiment 8 includes the features of any of embodiments 1-7, however the elongate core has a first stiffness, the outer layer has a second stiffness, and wherein the first stiffness is greater than the second stiffness.

Embodiment 9 includes the features of any of embodiments 1-8, however the first material is polyamide.

Embodiment 10 includes the features of any of embodiments 1-9, however the second material is polypropylene.

Embodiment 11 includes the features of any of embodiments 1-10, however the first material is at least about 50% of the elongate core by weight.

Embodiment 12 includes the features of embodiment 11, however the first material is at least about 60% of the elongate core by weight.

Embodiment 13 includes the features of embodiment 12, however the first material is at least 65% of the elongate core by weight.

Embodiment 14 includes the features of any of embodiments 9-13, however the first material also comprises an impact modifier.

Embodiment 15 includes the features of embodiment 14, however the impact modifier comprises at least about 10% of the first material.

Embodiment 16 includes the features of embodiment 15, however the impact modifier comprises at least about 15% of the first material.

Embodiment 17 includes the features of embodiment 16, however the impact modifier comprises at least about 20% of the first material.

Embodiment 18 includes the features of embodiment 17, however the impact modifier comprises at least about 25% of the first material.

Embodiment 19 includes the features of any of embodiments 14-18, however the impact modifier comprises styrene-ethylene-butylene-styrene.

Embodiment 20 includes the features of any of embodiments 1-19, however the first and second materials are co-extruded.

Embodiment 21 includes the features of any of embodiments 1-20, however the outer layer is extruded over the elongate core.

Embodiment 22 is an article. The article has a substrate. The substrate comprises an extruded mixture of a first material and a second material. The article also includes an outer layer comprising a third material. The outer layer at least partially covers an outer surface of the substrate. The third material comprises an activatable foaming agent. The activatable foaming agent undergoes thermal bonding with the second material during activation.

Embodiment 23 includes the features of embodiment 22, however the article is an earplug.

Embodiment 24 includes the features of either embodiment 22 or 23, however the foaming agent is configured to expand during activation.

Embodiment 25 includes the features of embodiment 24, however the foaming agent is configured to expand to take the shape of a mold during activation.

Embodiment 26 includes the features of any of embodiments 22-25, however the first material comprises polyamide and the second material comprises a polyolefin.

Embodiment 27 includes the features of embodiment 26, however the polyolefin is polypropylene.

Embodiment 28 includes the features of any of embodiments 22-27, however the first material is at least about 50% of the extruded mixture.

Embodiment 29 includes the features of any of embodiments 22-28, however the first material is at least about 60% of the extruded mixture.

Embodiment 30 includes the features of any of embodiments 22-29, however the first material is at least about 65% of the extruded mixture.

Embodiment 31 includes the features of any of embodiments 22-30, however the first material also comprises an impact modifier.

Embodiment 32 includes the features of embodiment 31, however the impact modifier is at least about 10% of the first material.

Embodiment 33 includes the features of embodiment 31, however the impact modifier is at least about 15% of the first material.

Embodiment 34 includes the features of embodiment 31, however the impact modifier is at least about 20% of the first material.

Embodiment 35 includes the features of embodiment 31, however the impact modifier is at least about 25% of the first material.

Embodiment 36 includes the features of any of embodiments 31-35, however the impact modifier is styrene-ethylene-butylene-styrene.

Embodiment 37 includes the features of any of embodiments 22-36, however the third material is extruded over the substrate.

Embodiment 38 includes the features of any of embodiments 22-37, however the outer layer forms a portion of a hearing protection device.

Embodiment 39 includes the features of any of embodiments 22-38, however the outer layer forms a portion of a facepiece seal.

Embodiment 40 includes the features of any of embodiments 22-39, however the outer layer forms a portion of a respiratory protection device.

Embodiment 41 includes the features of any of embodiments 22-40, however the outer layer forms a portion of an ear muff.

Embodiment 42 includes the features of any of embodiments 22-41, however the outer layer forms a portion of an eyewear article.

Embodiment 43 is a method of making an article. The method includes the step of forming a preform with a first end portion and a second end portion opposite of the first end portion by covering a substrate with an outer layer. The substrate comprises a first material coextruded with a second material. The outer layer comprises a third material and the third material extends at least partially from the first end portion to the second end portion. The method also includes the step of positioning the first end portion in a first mold cavity and the second end portion in a second mold cavity, the first mold cavity having a diameter that is smaller than a diameter of the second mold cavity at its widest point. The method also includes the step of applying heat to at least a portion of the outer layer of the second end portion such that at least a portion of the outer layer expands and conforms to a shape of the second mold cavity and the outer layer thermally bonds to the substrate, and such that the first end portion forms a stem and the second end portion forms a sound-attenuating portion.

Embodiment 44 includes the features of embodiment 43, however the third material comprising an unactivated expandable sphere foaming agent.

Embodiment 45 includes the features of embodiment 43 or 44, however applying heat comprises subjecting the portion of the outer layer to a temperature of between 100° C. to 205° C.

Embodiment 46 includes the features of any of claims 43-45, however thermal bonding comprises the second material bonding to the third material.

Embodiment 47 includes the features of any of claims 43-46, however the third material comprises unactivated chemical foaming agent.

Embodiment 48 includes the features of any of claims 43-47, however the first material comprises polyamide.

Embodiment 49 includes the features of any of claims 43-48, however the first material comprises an impact modifier.

Embodiment 50 includes the features of any of claims 43-49, however the second material comprises polypropylene.

Embodiment 51 includes the features of any of embodiments 43-50, however the first material comprises a scaffold structure within the substrate, and the second material comprises at least some discrete domains within the scaffold structure.

Embodiment 52 includes the features of any of embodiments 43-50 1 comma however the first material comprises at least 50 percent of the substrate.

Embodiment 53 includes the features of any of embodiments 43-52, however the first material comprises at least 60% of the substrate.

Embodiment 54 includes the features of any of embodiments 43-53, however the first material comprises at least about 65% of the substrate.

Embodiment 55 includes the features of any of embodiments 49-54, however the impact modifier comprises at least about 10% of the first material.

Embodiment 56 includes the features of any of embodiments 49-54, however the impact modifier comprises at least about 15% of the first material.

Embodiment 57 includes the features of any of embodiments 49-54, however the impact modifier comprises at least about 20% of the first material.

Embodiment 58 includes the features of any of embodiments 49-54, however the impact modifier comprises at least about 25% of the first material.

Embodiment 59 includes the features of any of embodiments 43-58, however the outer layer forms a portion of a hearing protection device.

Embodiment 60 includes the features of any of embodiments 43-58, however the outer layer forms a portion of a facepiece seal.

Embodiment 61 includes the features of any of embodiments 43-58, however the outer layer forms a portion of a respiratory protection device.

Embodiment 62 includes the features of any of embodiments 43-58, however the outer layer forms a portion of an ear muff.

Embodiment 63 includes the features of any of embodiments 43-58, however the outer layer forms a portion of an eyewear article.

Embodiment 64 is a method of making an article. The method includes the step of covering a substrate with an outer layer to prepare a preform having a first end portion and a second end portion. The substrate comprises a first material and a second material and the outer layer comprises a third material, the third material comprising a foaming agent having an activation temperature. The third material has a temperature below the activation temperature. The method also includes the step of positioning the first end portion of the preform in a first cavity of a mold and the second end portion in a second cavity of the mold, the first mold cavity having a diameter that is smaller than a diameter of the second mold cavity at its widest point. The method also includes the step of increasing the temperature of the second end portion to at least the activation temperature of the foaming agent to activate the foaming agent within the second end portion to form the second end portion into a sound-attenuating portion.

Embodiment 65 includes the features of embodiment 64, however the first material is polyamide.

Embodiment 66 includes the features of either embodiment 64 or 65, however the first material comprises an impact modifier.

Embodiment 67 includes the features of embodiment 66, however the impact modifier comprises at least about 10% of the first material.

Embodiment 68 includes the features of embodiment 66, however the impact modifier comprises at least about 15% of the first material.

Embodiment 69 includes the features of embodiment 66, however the impact modifier comprises at least about 20% of the first material.

Embodiment 70 includes the features of embodiment 66, however the impact modifier comprises at least about 25% of the first material.

Embodiment 71 includes the features of any of embodiments 64-70, however the first and second materials are coextruded.

Embodiment 72 includes the features of any of embodiments 64-71, however the first material is at least about 50% of the substrate.

Embodiment 73 includes the features of any of embodiments 64-72, however the first material is at least about 60% of the substrate.

Embodiment 74 includes the features of any of embodiments 64-73, however the first material is at least about 65% of the substrate.

Embodiment 75 includes the features of any of embodiments 64-74, however covering a substrate comprises extruding the third material over the substrate.

Embodiment 76 includes the features of any of embodiments 64-75, however the second material is a polyolefin.

Embodiment 77 includes the features of any of embodiments 64-75, however the second material is polypropylene.

Embodiment 78 includes the features of any of embodiments 64-77, however activating the foaming agent causes the second end portion to expand and to conform to a shape of the second cavity.

Embodiment 79 includes the features of any of embodiments 64-78, however the activation temperature is lower than a melting point of the first material.

Embodiment 80 includes the features of any of embodiments 64-79, however the activation temperature is high enough for thermal bonding between the second material and the third material.

Embodiment 81 includes the features of any of embodiments 64-80, however the activation temperature is between 100° C. and 205° C.

Embodiment 82 includes the features of any of embodiments 64-81, however it also includes the step of positioning the first cavity above the second cavity during activating of the foaming agent.

### EXAMPLES

### Core Test Methods

### Core Bond Test

Bonding between the outer foam material and the core is assessed visually from the appearance of the core after the foam molding process. In particular, the foam should be physically attached to the core after molding. The image on the right in 10A demonstrates a good foam bond. In this image, part of the foam remains adhered to the core when the outer foam material is torn away from the core. This indicates the mode of failure is cohesive failure of the foam. The image on the left shows a similarly shaped core after molding. In this case, when the foam is torn away, no foam remains bound to the core. This is considered an unacceptable failure.

### Core Mechanical Stability Test

Mechanical stability of the core is also assessed somewhat qualitatively. Measurements of the diameter and length of the core are made prior to molding. Failure occurs when the overall length or diameter of core changes by more than 5% after molding, as assessed using a caliper.

### Core Flexibility Test

Flexibility is related to the flexural modulus of the material used in the core, and is not thought to be affected by the molding process. However, highly rigid materials may be uncomfortable for the end user, and thus may be rejected for this application.

### EXAMPLE

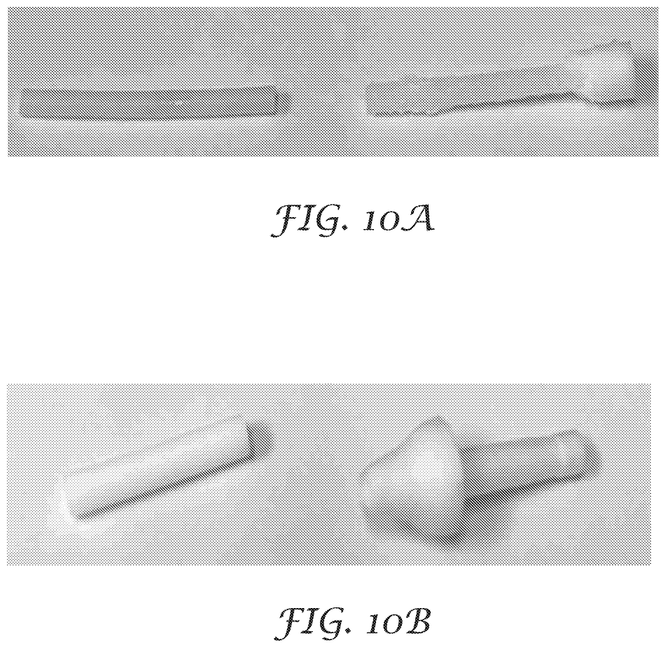

One inch cylindrical preforms shown on the left in FIG. 10B were made by extruding foamable material over a polypropylene core, then cutting the resultant overcoat extrudate to one inch lengths. To evaluate different core materials the polypropylene was pushed out of the middle of the pre-form and replaced with a similarly sized piece of alternative core material. Alternative core materials were either obtained as rod-stock from commercial sources and then cut to length or obtained in pellet form, then extruded and cut to length. The polypropylene core containing very long glass fiber was obtained via compression molding of preforms of approximately one inch length.

With the alternative core material in place, the preform was placed in a mold at 375 F for 5 minutes, then cooled to yield an earplug (example shown on the right in FIG. 2). The foam was then torn away from the core material and the bonding and deformation of the core was assessed as described above. Results for the various materials tested are shown in Table 1.

TABLE 1

| | Deformation | Bonding |
|---|---|---|
| Core Material | (pass/fail) | (pass/fail) |
| HDPE | fail | pass |
| Polypropylene (PP) | fail | pass |
| Crosslinked Polystyrene | pass | fail |
| Thermoplastic Polyurethane | fail | fail |
| Acetal | pass | fail |
| ABS | fail | fail |
| Polyamide | pass | fail |
| PETG | fail | fail |
| Polycarbonate | fail | fail |
| PP (10% short glass fiber filled) | fail | pass |
| PP (20% short glass fiber filled) | fail | pass |
| PP (10% very long glass fiber filled) | fail | pass |
| Crosslinked Polyethylene | pass | fail |
| PP (40% talc-filled) | fail | pass |
| PP-polyamide alloy | pass | pass |

1. A push-in earplug comprising:
an elongate core comprising a first material and a second material;
an outer layer comprising a foamable material, the outer layer covering at least a portion of an outer surface of the elongate core; and
wherein the second material is configured to thermally bond to the outer layer during activation of the foamable material.

2. The push-in earplug of claim 1, wherein the foamable material comprises a foaming agent that, when activated, increases volume of outer layer.

3. The push-in earplug of claim 2, wherein the foaming agent comprises an unactivated expandable sphere foaming agent.

4. The push-in earplug of claim 2, wherein the foaming agent comprises unactivated chemical foaming agent.

5. The push-in earplug of claim 1, wherein the foaming agent is heat activated.

6. The push-in earplug of claim 5, wherein the foaming agent is heat activated at a temperature that allows for thermal bonding with the second material, and wherein, the temperature is less than a melting temperature of the first material.

7. (canceled)

8. The push-in earplug of claim 1, wherein the elongate core has a first stiffness, the outer layer has a second stiffness, and wherein the first stiffness is greater than the second stiffness.

9.-13. (canceled)

14. The push-in earplug of claim 1, wherein the first material also comprises an impact modifier.

15-19. (canceled)

20. The push-in earplug of claim 1, wherein the first and second materials are co-extruded.

21. The push-in earplug of claim 1, wherein the outer layer is extruded over the elongate core.

22. An article comprising:
a substrate comprising an extruded mixture of a first material and a second material;
an outer layer comprising a third material, wherein the outer layer at least partially covers an outer surface of the substrate; and
wherein the third material comprises an activatable foaming agent, and wherein the activatable foaming agent undergoes thermal bonding with the second material during activation.

23. The article of claim 22, wherein the article is an earplug.

24. The article of claim 22, wherein the foaming agent is configured to expand during activation.

25. The article of claim 24, wherein the foaming agent is configured to expand to take the shape of a mold during activation.

26. The article of claim 22, wherein the first material comprises polyamide and the second material comprises a polyolefin.

27-30. (canceled)

31. The article of claim 22, wherein the first material also comprises an impact modifier.

32-36. (canceled)

37. The article of claim 22, wherein the third material is extruded over the substrate.

38-42. (canceled)

43. A method of making an article, comprising:
forming a preform with a first end portion and a second end portion opposite of the first end portion by covering a substrate with an outer layer, wherein the substrate comprises a first material coextruded with a second material, and wherein the outer layer comprises a third material, and wherein the third material extends at least partially from the first end portion to the second end portion;
positioning the first end portion in a first mold cavity and the second end portion in a second mold cavity, the first mold cavity having a diameter that is smaller than a diameter of the second mold cavity at its widest point; and
applying heat to at least a portion of the outer layer of the second end portion such that at least a portion of the outer layer expands and conforms to a shape of the second mold cavity and the outer layer thermally bonds to the substrate, and such that the first end portion forms a stem and the second end portion forms a sound-attenuating portion.

44. (canceled)

45. (canceled)

46. The method of claim 43, wherein thermal bonding comprises the second material bonding to the third material.

47-50. (canceled)

51. The method of claim 43, wherein the first material comprises a scaffold structure within the substrate, and wherein the second material comprises at least some discrete domains within the scaffold structure.

52-82. (canceled)
